(19) United States
(12) Patent Application Publication
(10) Pub. No.: US 2010/0021945 A1
(43) Pub. Date: Jan. 28, 2010

(54) DETERMINATION OF CHANGES IN CONCENTRATION

(75) Inventors: Werner Nau, Bremen (DE); Huseyin Bakirci, Strasbourg (FR); Andreas Hennig, Lengede (DE)

Correspondence Address: SUGHRUE MION, PLLC, 2100 PENNSYLVANIA AVENUE, N.W., SUITE 800, WASHINGTON, DC, 20037, US

(73) Assignee: JACOBS UNIVERSITY BREMEN GGMBH, Bremen (DE)

(21) Appl. No.: 12/301,145

(22) PCT Filed: May 10, 2007
(86) PCT No.: PCT/EP2007/004139

§ 371 Date: Sep. 17, 2009

(30) Foreign Application Priority Data
May 16, 2006 (DE) 10 2006 023 083.3

(51) Int. Cl. C12Q 1/527; C12M 1/34

(52) U.S. Cl. 435/7.92; 435/288.7

(57) ABSTRACT

The invention relates to the field of macrocyclic host systems and fluorescent dyes. In particular, the invention relates to apparatuses and methods for determining a change in the concentration of an analyte, in particular as a result of a catalysed reaction, preferably an enzymatically catalysed reaction and preferably in an aqueous solution (>50% by weight water).

The invention relates to the field of macrocyclic host systems and fluorescent dyes. In particular, the invention relates to apparatus and methods for determining a change in the concentration of an analyte, due in particular to a catalysed reaction, and preferably an enzymatically catalysed reaction, preferably in aqueous (water content >50% by weight) solution.

To allow biochemical reactions to be characterised, it is often necessary for changes in the concentration of co-factors in reactions to be determined, and in particular changes in the concentration of products or educts of reaction. What is usually selected for this purpose is a co-factor in the reaction in the form of an analyte, whose change in concentration during the course of the reaction which is to be characterised is determined. The concentration of the analyte is usually determined (a) by measuring a property, which is specific to the analyte in the given reaction, of the analyte as a substance or (b) by converting the analyte by means of a further substance whereby a specific, detectable change in the further substance is obtained. For example, the concentration of an analyte may be determined by a procedure of the (a) type by looking at its fluorescence. A disadvantage in this case is that only a few substances are suitable for detection by fluorescence spectroscopy, which means that there are only a few reactions for which this method can be performed. Because of this, the concentration of an analyte is usually determined by a procedure of the (b) type such for example as by binding the analyte to an antibody and by determining the proportion of antibodies which have bound to the analyte as a proportion of the total number of antibodies which were available. Detection of this kind may for example be performed by means of the Biacore system. A disadvantage that has been found in this case is that when the analyte concentration varies rapidly the analytes cannot be bound or released quickly enough by the antibodies which bind them. This generally makes it necessary for there to be waiting times (what are referred to as incubation times) of around 20 minutes and these routinely prevent repeated measurements at short intervals, or in other words a continuous measurement, from being made (see for example Geymayer, P., Bahr, N & Reymond, J.-L. Chem. Eur. J. 5, 1006-1012 (1999)).

To allow biochemical reactions to be characterised, an attempt is therefore generally made to determine the concentration of the analyte to be analysed by doing so directly, without the mediation of other substances which may be required for the detection and without any chemical change to the analyte. A disadvantage in this case is that for many analytes special, individual analytical procedures have to be developed because for example the chemical structures of the analytes, and hence their chemical and physical properties, differ from one another.

Attempts have therefore been made to make analytes needing to be detected more easily detectable by providing them with a detectable group such for example as a fluorophor. For this purpose, the analyte is covalently connected to a fluorescent group before the reaction is performed. When the reaction is performed and the analyte is converted in the appropriate way, the intention is that the fluorescence of the fluorescent group will be changed, for example by the splitting off of the group or by a change in the structure of the analyte. The change in fluorescence is then a measure of the conversion of the analyte.

However, there is a disadvantage with this, which is that the fluorescent group often affects the reactance of the analyte. Particularly with enzymatically catalysed reactions, it is possible that the fluorescent group may prevent the analyte from binding to or detaching from the enzyme or may make this more difficult, which means that there will be errors in the characterising of the reaction to be characterised, or rather in the characterising of the enzyme which is to be characterised by looking at the reaction. Another disadvantage is the amount of labour which is involved in the covalent linking that has to be performed of the analyte to the fluorescent group.

It was therefore an object of the present invention to specify a method of determining, in a specimen, a change in the concentration of an analyte, which method avoids or mitigates the above disadvantages. In particular, the method should be capable of being applied to a plurality of analytes, it should be easy to perform, it should allow both slow changes in concentration (k_{cat} in the range from 5 to 10⁴ s⁻¹ and preferably in the range from 5 to 15 s⁻¹) and also fast changes in concentration (k_{cat} higher than 10⁴ s⁻¹, and preferably in the range from 10⁵ to 10⁷ s⁻¹ and, as a particular preference, in the range from 5*10⁵ to 5*10⁶ s⁻¹) to be determined rather than merely allowing analyte concentrations to be determined at equilibrium points of the reaction, it should make it possible for the course of the reaction to be followed on-line with no appreciable delay, it should be able to be performed with the option of, but not with the necessity for, the taking of samples from the reaction as it progresses, and/or it should be easily adaptable to a high-throughput method of analysis.

A further object was to specify an apparatus with which the method according to the invention can be performed. In particular, the apparatus should be easy to operate, should make it possible for changes in the concentration of analytes to be determined quickly, should make on-line determination of changes in the concentration of analytes possible and/or should be suitable for the carrying out of high-throughput methods of testing and, as well as this, should make swift determination possible for large numbers of specimens.

In accordance with the invention, a method of determining, in a specimen, a change in the concentration of an analyte during a reaction is therefore specified, comprising the steps of:
a) providing a fluorescent dye and a macrocycle for the specimen to be examined, the macrocycle being able to bind the dye and the analyte being able, in the range of concentrations of the analyte which are to be examined, to remove the fluorescent dye from the macrocycle, and
b) measuring a fluorescent property of the fluorescent dye at least two points in time.

The invention thus makes it possible, in a particularly advantageous manner, for the change in the concentration of an analyte in a specimen to be determined while a reaction is being performed, without any chemical change to the analyte and in particular without any covalent attachment of a fluorescent group. In so doing, the method according to the invention decouples the substances taking part in reaction which is to be characterised in an advantageous and surprising way from the substances which are used to give evidence of the change in the concentration of the analyte. The determination of the change in analyte concentration is thus made possible without an analyte having to make a sustained covalent bond to another substance or a bond which would affect the kinetics of the reaction which is being examined. In a surprising simple and advantageous way, the method according to the invention thus reduces a possible source of errors which might otherwise falsify or interfere with the characterisation of a reaction and the determination of a change in analyte concentration. If the analyte concentration changes as a result of a chemical conversion, it is useful in this case, and is preferred in all the embodiments of the invention which are described below, for an appropriate product of the conversion of the analyte, or an appropriate precursor (educt) of the analyte, not to remove, or not to be able to remove, the fluorescent dye from the macrocycle, or not to do this or to be able to do this as powerfully as the analyte. For the invention in all its aspects it is therefore preferred for the precursor (educt) of the analyte, or the product of the conversion of the analyte, to have a lower binding constant to the macrocycle than the analyte; the said binding constant is preferably lower than that of the analyte by at least a factor of 1.5 and, as a particular preference, is lower than it by a factor of 10 to 1000. The binding constant can be determined in, in particular, the manner described in Adv. Funct. Mater. 2006, 16, 237-242.

The values of the particular fluorescent property or properties which are measured at the two points in time are preferably used to calculate a variable which is representative of the difference in the fluorescent property. Fluorescent properties which are preferably to be measured are: intensity of fluorescence as a fluorescent property which is a particular preference, fluorescence life-time, polarisation of fluorescence, spectrum of fluorescence, and absorption at a preselected wavelength. Fluorescence life-time is preferably determined in the manner described in chapters 4 and 5 of Lakowicz, J. R., Principles of Fluorescence Spectroscopy, Kluwer Academic/Plenum: New York, 1999. It is also possible in accordance with the invention for two or more of the properties mentioned to be measured, and to be analysed to allow a change in the concentration of the analyte to be determined.

What is preferably determined is the difference between or the ratio to one another of the values of the fluorescent property at the first and second points in time, such for example as the difference between the first and second intensities of fluorescence or the ratio of the second intensity of fluorescence to the first intensity of fluorescence, although what are also meant are any other desired methods of calculating a representative variable.

For the purposes of the present invention, a fluorescent dye is a colorant which is able to absorb ultraviolet radiation or visible light and to emit it as light of a longer wavelength with virtually no delay (fluorescently). Fluorescent dyes for the purposes of the present invention are both organic, hetero-organic or organometallic dye molecules and also chromophoric components (fluorochromes) of larger molecular units and also luminescent lanthanide ions; a good survey of common fluorescent dyes and their fields of use can be found, in accordance with the invention, in the Handbook of Fluorescent Probes and Research Chemicals, Richard P. Haugland, Molecular Probes (Invitrogen).

A macrocycle for the purposes of the invention is a compound in ring form which is able to complex the fluorescent dye in a vacant internal space while changing its fluorescent properties. Preferred macrocycles for the purposes of the present invention are calixarenes, cucurbiturils, and cyclodextrins. Macrocycles which are a particular preference, and in particular preferred calixarenes, cucurbiturils, and cyclodextrins, will be described at a later point in the description.

An analyte for the purposes of the present invention is a substance which takes part in the reaction which is to be examined as a co-factor. The analyte may in particular be a product of reaction or an educt of reaction. In the course of the reaction which is being examined, the chemical structure of the analyte is changed. In preferred embodiments of the invention it is the charge, constitution or configuration of the analyte which is changed. The analyte is preferably an organic or hetero-organic compound. The analyte preferably consists of or comprises elements covalently connected to one another which are selected from the group comprising carbon, oxygen, nitrogen, phosphorus, sulphur and hydrogen.

In accordance with the invention, the fluorescent dye and the macrocycle are matched to the analyte whose change in concentration is to be determined. The fluorescent dye is complexed by the macrocycle by a relationship of the host-guest type, the fluorescent properties, and in particular one or more of the fluorescent properties listed above, of the fluorescent dye which is complexed being changed in comparison with its corresponding properties in free solution. The analyte may remove the fluorescent dye from the macrocycle. Quantities of fluorescent dye of different sizes are therefore released from the molecules of the macrocycle depending on the concentration of the analyte. In the range of concentrations which are to be examined, the fluorescence of the fluorescent dye is thus dependent on the concentration of the analyte without the fluorescent dye being covalently connected to the analyte or the analyte having to make some other connection to the fluorescent dye. In embodiments of the invention which are a particular preference, the analyte, the fluorescent dye and the macrocycle are each individual molecules which are not connected together covalently.

What are known to date from the prior art are only methods for detecting inorganic or organic cations with sulphonato-calix[4]arene by removing a fluorescent azoalkane from the calixarene (Bakirci et al., Chem. Commun., 2005, 5411 to 5413, and Adv. Funct. Mater. 2006, 16, 237-242). U.S. Pat. No. 6,475,803 B1 also discloses a method of checking whether at least a desired one of seven organic compounds is contained in drinking water, a fluorescent group being covalently attached to a cyclodextrin. Both methods merely disclose the possibility of determining a preset concentration. What they do not disclose on the other hand is that it would be possible for a change in the concentration of an analyte to be determined during a reaction and in particular during a catalysed, and in particular enzymatically catalysed, reaction. What in particular is not apparent from the publications cited is the speed at which the change in fluorescence would be able give a picture of a change in the concentration of the cation.

In accordance with the invention, it has now been appreciated for the first time that there is no need for a strong chemical interaction, and in particular a covalent connection, between the analyte whose change in concentration is to be determined and a substance which indicates the change in concentration, and instead, in the method according to the invention, a change in concentration is made capable of being detected, surprisingly quickly, by the mediation of a macrocycle as a result of an analyte removing the fluorescent dye from the macrocycle.

For the first time, the method according to the invention thus makes possible specific on-line monitoring of a reaction which is taking place, without any appreciable delay and for a plurality of analytes and without any complicated and effortful synthesis of fluorophor and analyte fusion molecules being required beforehand.

The change in the concentration of the analyte can for example be detected if the analyte is an educt of reaction which is converted in the course of the reaction into one or more products which are no longer able to remove the fluorescent dye from the macrocycle. In this case the concentration of the fluorescent dye in free solution is comparatively high at the beginning of the reaction because the analyte removes a large amount or all of the fluorescent dye from the molecules of the macrocycle. In the course of the reaction the concentration of the analyte goes down, and a higher proportion of the molecules of the fluorescent dye are thus able to complex with the molecules of the macrocycle. There is accordingly a change in the fluorescence of a specimen which is being examined and in which the reaction which is being examined takes place.

If the analyte is a product of reaction, then at the beginning of the reaction there is a comparatively high proportion of the molecules of the fluorescent dye which are complexed with molecules of the macrocycle. In the course of the reaction, an increasingly high proportion of the molecules of the fluorescent dye are removed from the molecules of the macrocycle, and the fluorescence of a specimen in which the reaction to be examined takes place changes accordingly.

Some particular guidelines follow which the person skilled in the art can use to guide him in selecting fluorescent dyes and macrocycles:

When the analyte whose change in concentration is to be determined is an anion, then the macrocycle is usefully an anion receptor. For anionic analytes the macrocycle is preferably a calixarene or cyclophane having cationic groups. A macrocycle which is able to complex anions, and in particular a calixarene having cationic groups, is therefore suitable in an advantageous way for use as a macrocycle in a reaction in which (a) an anion is converted into an uncharged molecule or an uncharged molecule is converted into an anion and (b) an anion is converted into a different anion which has a different charge or is of a different size or a different form (constitution or configuration). When the analyte is an anion, then the fluorescent dye too is preferably an anion.

When the analyte whose change in concentration is to be determined is a cation, then the macrocycle is usefully a cation receptor. For cationic analytes the macrocycle is preferably a calixarene or cyclophane having anionic groups or a cucurbituril. A macrocycle which is able to complex cations, and in particular a cucurbituril, or a calixarene or cyclophane having anionic groups, is therefore suitable in an advantageous way for use as a macrocycle in a reaction in which (a) a cation is converted into an uncharged molecule or an uncharged molecule is converted into a cation and (b) a cation is converted into a different cation which has a different charge or is of a different size or a different form (constitution or configuration). When the analyte is a cation, then the fluorescent dye too is preferably a cation.

When the analyte is able to enter into a hydrogen-bridge bond then the macrocycle is preferably a macrocycle having the ability to form hydrogen-bridge bonds. Preferred macrocycles having the ability to form hydrogen-bridge bonds are cyclodextrins. Macrocycles having the ability to form hydrogen-bridge bonds, and in particular cyclodextrins, are therefore able to be used, with advantage, to determine a change in the concentration of an analyte in a reaction in which (a) a substance not capable of forming hydrogen-bridge bonds is converted into a hydrogen-bridge donor or acceptor forming an analyte, or vice versa, in which (b) a hydrogen-bridge donor is converted into a hydrogen-bridge acceptor, in which case the hydrogen-bridge donor or the hydrogen-bridge acceptor may be the analyte, or in which (c) an analyte capable of forming hydrogen bridges is converted into a substance of a different size or a different form (constitution or configuration). The fluorescent dye is preferably likewise a substance capable of forming hydrogen-bridge bonds either as a donor or acceptor as the case may be.

If the analyte is distinguished from other substances which take part in the reaction simply by its size then the macrocycle is preferably selected in such a way that it is able to complex substances of the same size as the analyte but not of the same size as a conversion product of the analyte or of the same size as a precursor into which the analyte is to be converted. The macrocycle may preferably be a calixarene, a cyclophane, a cucurbituril or a cyclodextrin in this case. The fluorescent dye is then usefully of a similar size to the analyte which complexes with the macrocycle.

If the analyte is to be distinguished from a precursor (educt) or a product of reaction stereo-selectively, the macrocycle is preferably a cyclodextrin of a size suitable for binding the analyte.

Preferred calixarene macrocycles are:

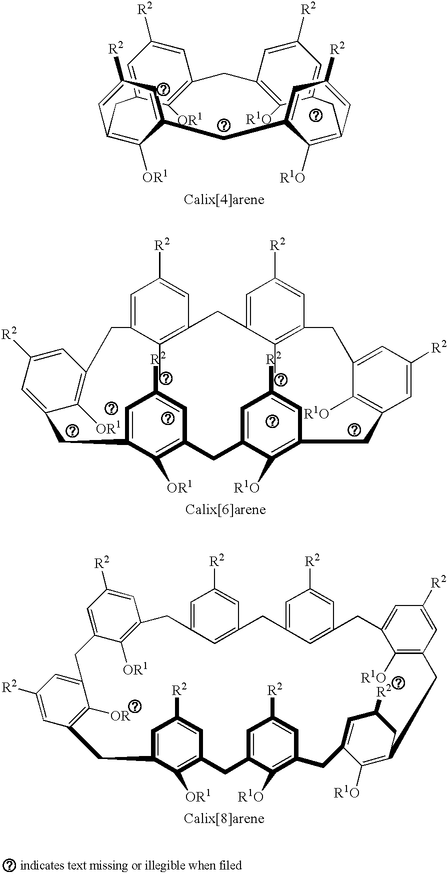

where, independently of one another,

R¹=H, (CH₂)ₘCH₃ where m is 0, 1, 2, 3, 4, 5, 6 or 7 or a branched or straight-chain C₁-C₈-alkyl or C₁-C₈ alkenyl and preferably isopropyl or tert-butyl,

R²=(CH₂)ₘX where m is 0, 1, 2 and X is F, Cl, Br, I, SO₃H, SH, OH, COOH, NH₂, NRH, NR₂, NR₃⁺ (in each case with R selected from methyl, ethyl, n-propyl and isopropyl).

A preferred cyclophane macrocycle is:

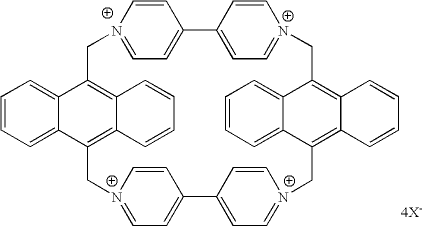

Where X⁻ is a counterion, preferably a chloride.

Preferred cyclodextrin macrocycles are:

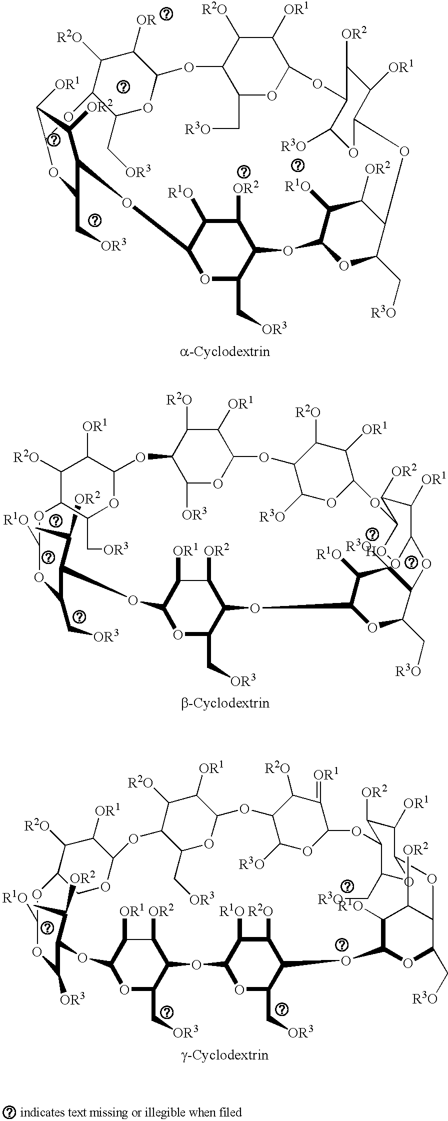

where, independently of one another, each R¹, R², R³ is selected from H, methyl, ethyl, n-propyl, isopropyl, butyl, octyl, (CH₂)_{1,2}COOH, (CH₂)_{2,3}OH, acetyl, benzoyl, sulpho, maltosyl, hydroxypropyl, succinyl and there may also be provided, in place of OR³, an NHR⁴, NR⁴R⁵ or (NR⁴R⁵R⁶)⁺ group where R⁴, R⁵ and R⁶ are in turn, independently of one another, selected from H, methyl, ethyl, n-propyl, isopropyl, butyl, octyl, (CH₂)_{1,2}COOH, (CH₂)_{2,3}OH, acetyl, benzoyl, sulpho, maltosyl, hydroxypropyl, succinyl.

Preferred cucurbituril macrocycles are:

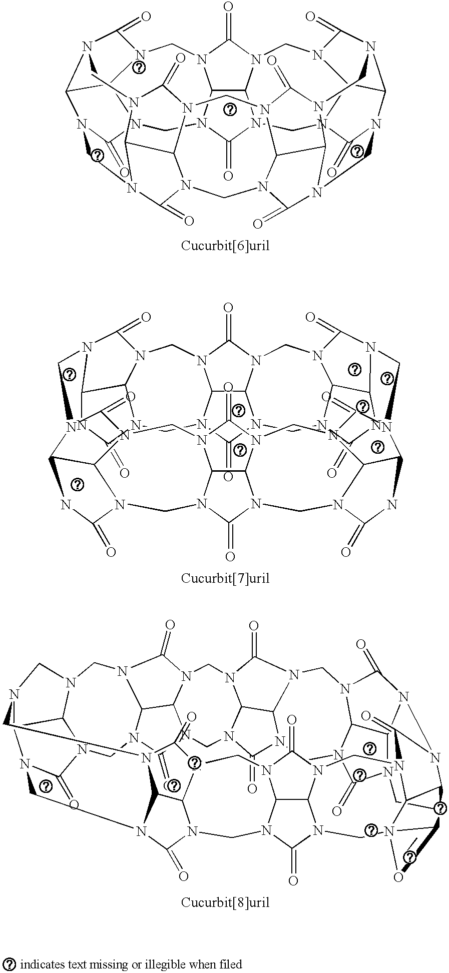

and, generally

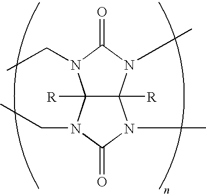

Where n is 6, 7, 8 and any R, independently of any other R, is selected from H, methyl and hydroxy.

The person skilled in the art will be able to put together suitable and preferred macrocycle/fluorescent dye pairings by employing one of the selection procedures described above. For many analytes, the macrocycle/fluorescent dye pairings given in Table I below are suitable and preferred:

TABLE I

| Macrocycle | Fluorescent dye |
|---|---|
| Calix[n]arene | DBO acetic acid |
| n = 4 | |
| R¹ = H, alkyl | |
| R² = (CH₂)ₘNR₂ where m = 1, 2, 3 and R = H, Me, Et | 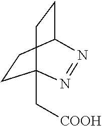 |
| Calix[n]arene n = 4, 6, 8 R¹ = H, alkyl R² = (CH₂)ₘNR₂ or (CH₂)ₘNR₃⁺, where m = 0, 1, 2 and R = H, Me, Et, allyl | 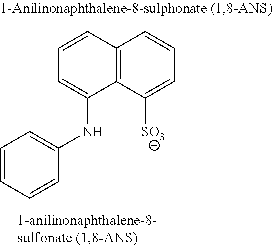 |
| Calix[n]arene n = 4 R¹ = H, alkyl R² = SO₃H | 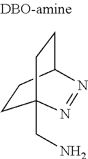 |
| Calix[n]arene n = 4, 6, 8 R¹ = H, alkyl R² = SO₃H | 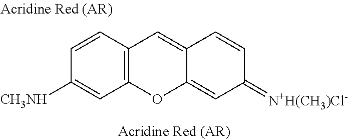 |
| Calix[n]arene n = 4, 6, 8 R¹ = H, alkyl R² = SO₃H | 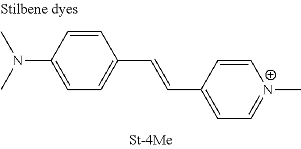 |
| | 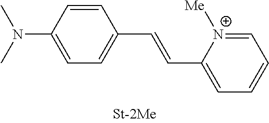 |
| | 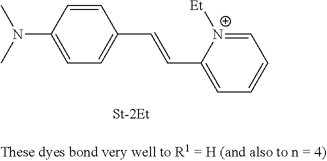 |
| Calix[n]arene n = 6, 8 R¹ = H, alkyl R² = SO₃H | 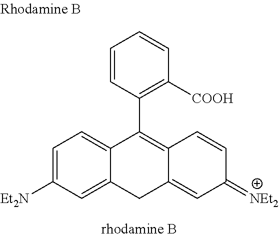 |
| Calix[n]arene n = 4, 6, 8 R¹ = H, alkyl R² = SO₃H | 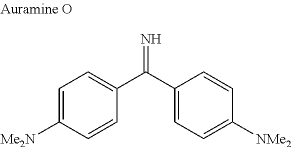 |
| Calix[n]arene n = 4, 6, 8 R¹ = H, alkyl R² = SO₃H | 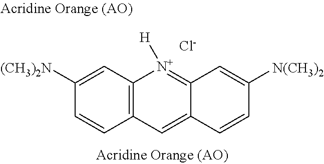 |
| Calix[n]arene n = 6, 8 R¹ = H, alkyl R² = SO₃H | 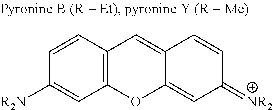 |

Preferred conditions for the above calixarene/fluorescent dye pairings: aqueous solution (including when buffered:
e.g. citrate, acetate or phosphate buffer), pH = 2 to 9

| | |
|---|---|
| 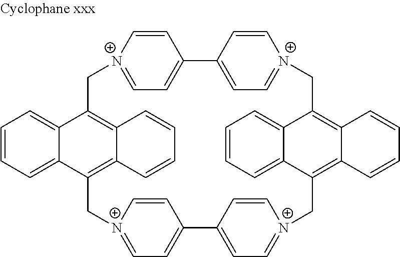 | 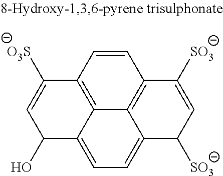 |
| 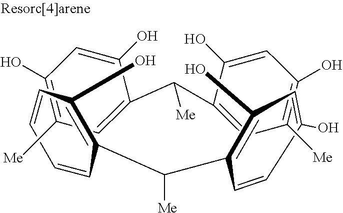 | 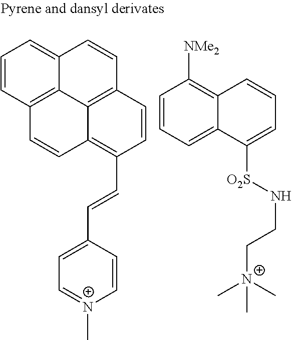 |

Preferred conditions: aqueous solution (including when buffered: e.g. citrate, acetate or phosphate buffer), pH = 2 to 12

| | |
|---|---|
| β-Cyclodextrin R¹ = R² = R³ = H | 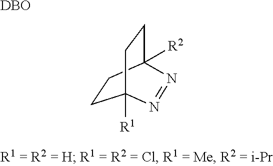 |
| β-Cyclodextrin R¹ = R² = R³ = H | 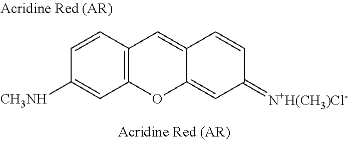 |
| β-Cyclodextrin R¹ = R² = R³ = H | 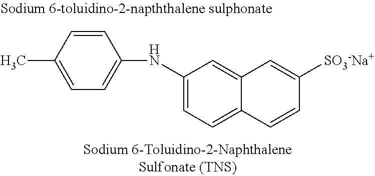 |
| β-Cyclodextrin R¹ = R² = R³ = H | 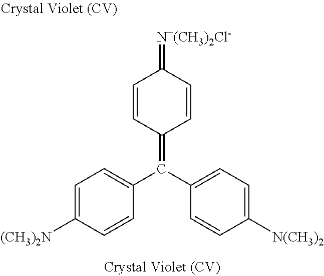 |
| β-Cyclodextrin R¹ = R² = R³ = H | 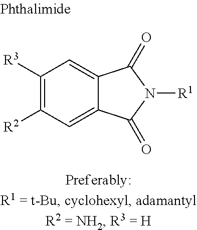 |
| β-Cyclodextrin R¹ = R² = R³ = H, Me, hydroxypropyl | 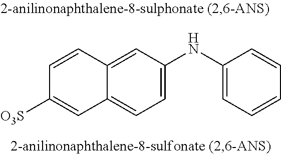 |
| β-Cyclodextrin R¹ = R² = R³ = H γ-Cyclodextrin R¹ = R² = R³ = hydroxypropyl | 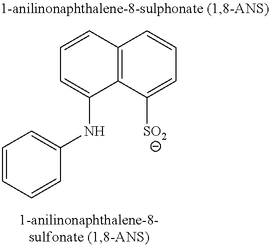 |
| α-Cyclodextrin R¹ = R² = R³ = H | 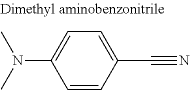 |
| β-Cyclodextrin R¹ = R² = R³ = H | 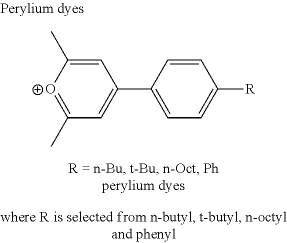 |

Preferred conditions for the cyclodextrin/fluorescent dye pairings: aqueous solution (including when buffered:
e.g. citrate, acetate or phosphate buffer), pH = 2 to 12

| | |
|---|---|
| Cucurbiturils | Cucurbituril/fluorescent dye pairings are specified in |
| | WO2006/005727, the entire disclosure content of |
| | which is hereby incorporated by reference for the |
| | purposes of the present invention. |
| Cucurbit[7]uril | 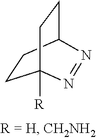 |
| Cucurbit[7]uril | 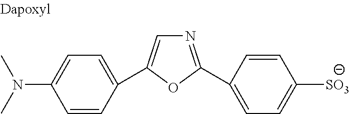 |
| Cucurbit[7]uril | Rhodamine 6G |
| Cucurbit[7]uril | Tetramethyl rhodamine |
| Cucurbit[7]uril | 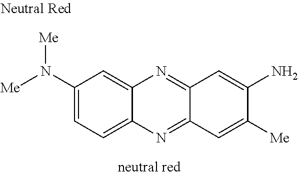 |
| Cucurbit[7]uril | 2-aminoanthracene |
| Cucurbit[7]uril | 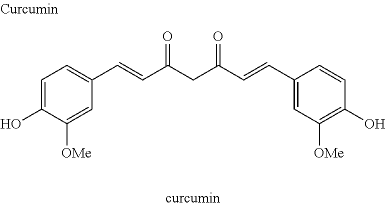 |
| Cucurbit[8]uril | 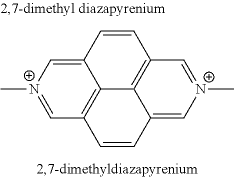 |
| 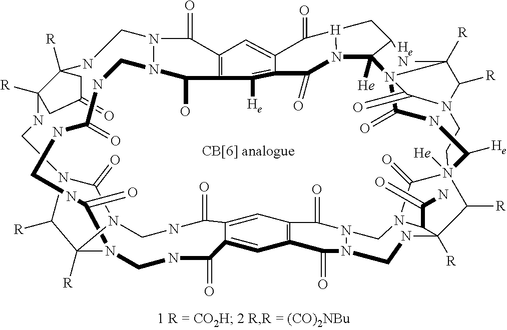 | 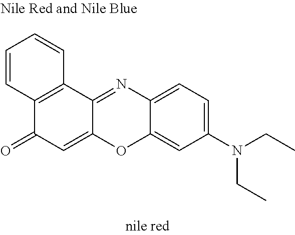 |
| | 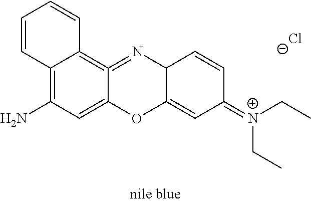 |

Preferred conditions for the cucurbituril/fluorescent dye pairingS: aqueous solution (including when buffered:
e.g. citrate, acetate or phosphate buffer), pH = 2 to 12

The concentration of the analyte preferably changes because the analyte takes part in a reaction in the course of which it is converted into a different substance, for example by being isomerised, by having one or more chemical groups added to it or taken away from it, or by having its charge altered. It is likewise preferred for the analyte to derive from an appropriate reaction in which a precursor substance (educt) is converted into the analyte in the manner just described.

A particular preference is a method according to the invention in which there is present in addition in the specimen a co-factor in the reaction to convert the analyte or to convert a precursor (educt) into the analyte. Most of the biochemically relevant reactions are reactions of this kind. The carrying out of a method according to the invention by following in particular the guidelines given above for selecting the macrocycle and fluorescent dye is therefore a particular preference in accordance with the invention.

It is particularly preferred in this case for the co-factor in the reaction to be a catalyst and in particular an enzyme. In accordance with the invention, the co-factor of the analyte or of the analyte precursor may thus derive unchanged from the reaction, as is usual for a catalyst or enzyme. Enzymes and reactions which are preferred in accordance with the invention are given in Table II below, remembering that in each case the analyte may be a product or educt of the reaction which is catalysed by the given enzyme and may include a co-factor.

TABLE II

| Class of enzyme | Preferred sub-classes and preferred enzymes |
|---|---|
| EC1 oxidoreductases | All except EC 1.9.x.x, 1.15.x.x, 1.18.x.x, |
| | 1.19.x.x and 1.21.x.x, though it is possible |
| | for the co-factor to be used as an analyte |
| | regardless of this, namely for the EC classes |
| | 1.x.1.x, 1.x.4.x, 1.x.5.x, 1.x.6.x, and in |
| | some cases for 1.x.98.x and 1.x.99.x. |
| EC 2 transferases | All |
| EC 3 hydrolases | All |
| EC 4 lyases | All |
| EC 5 isomerases | Particular preferences are EC 5.1.x.x, EC |
| | 5.2.x.x and EC 5.4.x.x to EC 5.99.x.x |
| EC 6 ligases | Particular preferences are EC 6.1.x.x, 6.2.x.x, |
| | 6.5.x.x and 6.6.x.x |

In an advantageously simple way, the method according to the invention makes it possible for enzymes to be characterised and, when this is done, makes it possible in each case for the choice made of the analyte to be largely a free one. In this way, the method according to the invention for the first time makes possible characterisation of an enzyme of a kind which would otherwise only be possible by the targeted selection of an analyte or by the covalent linking of an analyte to a detectable group which was for example fluorophoric, which characterisation could therefore could not be anything but complicated and effortful or highly subject to error.

Because of the versatile way in which it can be used, the method in according to the invention may, in an advantageous way, be included in a method of screening according to the invention. Therefore, a method of screening for finding a co-factor for an analyte is also provided in accordance with the invention, which method of screening comprises the steps of:
a) providing a plurality of mixtures each containing the preselected analyte or a precursor (educt) of the analyte, a reaction co-factor to be tested for producing the analyte from the precursor or for converting the analyte, a fluorescent dye, and a macrocycle, the analyte being able to remove the fluorescent dye from the macrocycle in the range of concentrations of the analyte which are to be examined, and, as an option, an inhibitor of the reaction, and
b) performing a method of determination according to the invention with each of the mixtures provided in a).

For each of the mixtures provided in a), the fluorescent property of the fluorescent dye, and in particular the intensity of fluorescence, fluorescence life-time, polarisation of fluorescence, spectrum of fluorescence, and/or absorption at a preselected wavelength, is therefore measured at least two points in time during the reaction which takes place in the mixture. The change in the fluorescent property of the mixture, such as in its intensity of fluorescence for example, is then a measure of the change in the concentration of the analyte. By comparing the changes in fluorescence in different mixtures, it is then possible for example to identify co-factors for the analyte which make it possible for the analyte to be converted quickly or a precursor to be converted into the analyte quickly. Such a method is therefore particularly suitable for use as a method of screening for co-factors in an enzymatically catalysed reaction. Respective preselected enzymes and respective specific analytes or analyte precursors (i.e. precursors which, as educts, will be converted into the analytes) may for example be made available in the mixtures to be examined, and a substance from a library of substances is then added and the change in the concentration of the analyte is determined. The analyte in a method of screening of this kind is then preferably a co-factor, and in particular is preferably adenosine triphosphate, adenosine diphosphate, adenosine monophosphate, cycloadenosine monophosphate, nicotinamide adenine dinucleotide (NAD+) and the appropriately reduced compound (NADH+H+), and nicotinamide adenine dinucleotide phosphate (NADP) and the appropriately reduced compound (NADPH+H+). By a method of screening of this kind it is also possible to find an inhibitor for a known reaction. The systematic search (the screening) for inhibitors for a preselected reaction is particularly important from an economic point of view, particularly for finding medicaments. What is done in this case is that the educt or educts from the reaction being considered is/are made available and a possible inhibitor of the reaction is added as a substance from the library of substances (the inhibitor library). The change in fluorescence, which is a measure of the change in the concentration of an analyte (which may be a product of reaction or an educt and in particular a co-factor as just mentioned) is then a measure of the inhibiting effect of the possible inhibitor substance which was selected from the library of substances. It is preferred for an inhibitor to be found for, once again, a catalysed, and in particular enzymatically catalysed, reaction to be found. The analyte may be one of the above-mentioned co-factors or some other product of reaction or educt in this case. In particular, it is now possible by the method according to the invention for inhibitors to be found for a reaction which is catalysed by one of the enzymes given in Table II. The method according to the invention is therefore particularly suitable for the development of medicaments in that it makes it easier, or for the first time makes it possible in practical terms, for pharmaceutically valuable inhibitors of a preselected reaction to be found in a large library of potential inhibitors (an inhibitor library). As a particular preference, the method is used to find inhibitors for a reaction which is catalysed in aqueous solution by one of the enzymes in table II.

The method of screening according to the invention is also suitable for investigating what catalyst is able to catalyse a given reaction. For this purpose, the educt or educts of the reaction are provided and a catalyst which is a possibility in the given case is selected from a library of substances (a catalyst library), and in particular an enzyme which is a possibility is selected from, for example, a library of mutations, and is added. The change in fluorescence is then a measure of the change in the concentration of an analyte, in which case the analyte may in turn be a product of reaction or an educt and in particular one of the co-factors mentioned above or some other product of reaction or educt. In this way it is for example possible to investigate what possible enzyme from a library or enzymes is able to catalyse a preselected reaction fastest or with the best yield.

The method according to the invention is also suitable for determining which catalyst from a library of substances (a catalyst library) is suitable for catalysing a given reaction in spite of the presence of an inhibitor. For this purpose, the educt or educts of reaction are once again provided, together with the inhibitor, and a catalyst which is a possibility from a library of substances, and in particular an enzyme from a library of enzyme mutants, is added. What is used as an analyte is, once again, a product of reaction or an educt and in particular one of the co-factors mentioned above. The change in fluorescence, as a measure of the change in the concentration of the analyte, then indicates the ability of the catalyst, and in particular of the enzyme, to catalyse the preselected reaction despite the presence of the inhibitor, and the extent of this ability.

The method according to the invention, and in particular a method of screening according to the invention, is therefore suitable, with particular advantage, for finding pharmaceutically effective constituents which are for example intended to make possible or prevent a preselected reaction. The method according to the invention calls, in this case, for only a minimum amount of adjustment to the reaction which is to be examined in the given case in that it is merely a macrocycle/fluorescent dye pairing which has to be matched to the analyte in the given case. If what is selected as the analyte is a substance which is repeatedly encountered in a plurality of biochemical reactions, such as an enzyme co-factor for example and in particular one of the co-factors mentioned above, then a standardised method according to the invention employing a preselected dye and macrocycle can be used. The method according to the invention is however also able to be used with particular advantage for screening a library of inhibitors, i.e. for investigating which substance is able to inhibit a preselected reaction particularly well, and is also particular well suited to screening a library of enzymes, i.e. for investigating which enzyme, i.e. which catalyst from a library of catalysts, is able to catalyse a given reaction particularly well when there is or is not an inhibitor. In such cases, a suitable fluorescent dye/macrocycle pairing needs to be selected only once and can then be used for screening in a large number of mixtures.

The method according to the invention, and in particular the method of screening according to the invention, is preferably a high-throughput method and in particular a high-throughput method of screening. High-throughput methods of this kind make it possible for large libraries of substances to be examined in a short time, usually on a standardised system which operates automatically, thus enabling errors in measurement to be kept to an advantageously low level. As part of a high-throughput method, and in particular a high-throughput method of screening, of this kind, at least multiples of 48 mixtures or specimens, and as a particular preference at least 96, 384 or 1536 mixtures or specimens (including where required appropriate control mixtures or control specimens), are examined. The method according to the invention is particular easy to automate and in this way enables a library of substances, and in particular libraries of catalysts, enzymes, inhibitors and/or other co-factors, to be examined in a short time with low measurement errors.

To carry out the method according to the invention, and in particular a method of screening according to the invention, an apparatus for determining a change in the concentration of an analyte is specified, this apparatus comprising:
a specimen containing a fluorescent dye and a macrocycle, the analyte being able to remove the fluorescent dye from the macrocycle in the range of concentrations which is to be examined of the analyte, and
a co-factor for converting the analyte.

The co-factor may in this case also convert a precursor (educt) of the analyte into the analyte. An apparatus of this kind in which an analyte is altered materially or a precursor of the analyte is altered materially into the analyte thus makes it possible, in a particularly simple way, for the advantages which were described above for a method according to the invention to be achieved. As in all the other aspects of the invention, so in this one too it is preferred for the precursor of the analyte or the product of the conversion of the analyte not to be able to remove the fluorescent dye from the macrocycle and not to be able to do so as powerfully as the analyte does.

The apparatus according to the invention also comprises a light source for exciting the fluorescent dye and a detector for measuring the fluorescence and/or light absorption emanating from the fluorescent dye. It is also preferred for the apparatus to comprise a carrier having receptacles for at least multiples of 48 samples or mixtures to be examined, the multiples preferably being 96, 384 or 1536 samples or mixtures which include, if required, intended control specimens or control mixtures. Carriers which are particularly preferred are so-called microtitre plates having an appropriate number of wells.

A significant advantage of the method according to the invention as compared with the use of antibodies is that the changes in concentration, and in particular changes in concentration which are brought about enzymatically, can be followed continuously.

The invention will be further described below by reference to an embodiment and the drawings, although by so doing it is not intended to limit the scope of protection claimed by the claims.

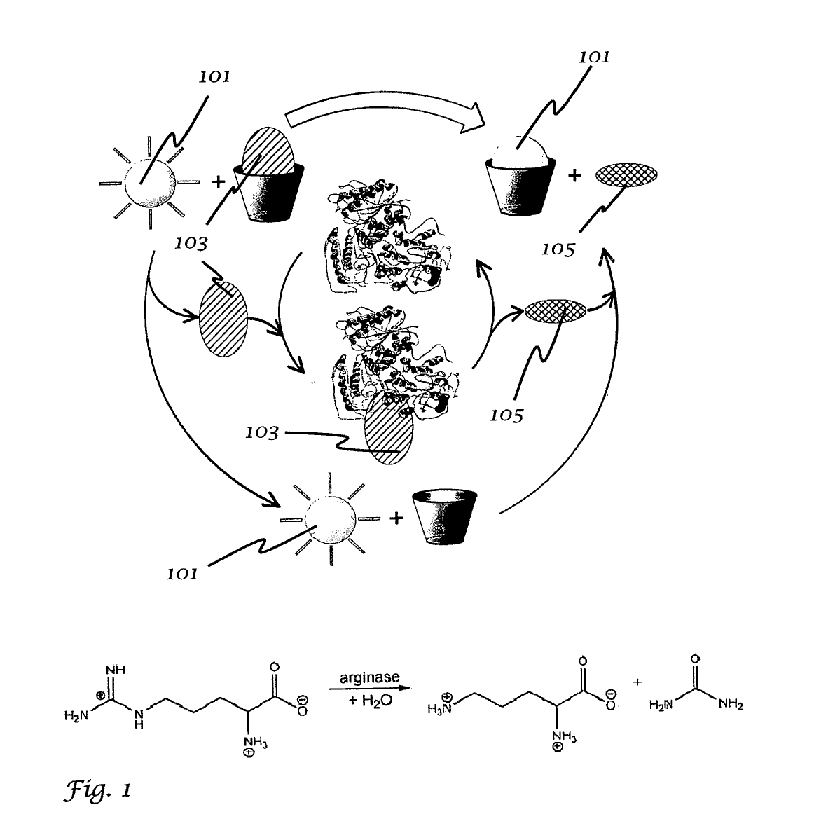

FIG. 1 is the reaction diagram for the conversion of L-arginine (103, analyte, which removes the fluorescent dye) into L-ornithine (105, product) and urea (not shown, product) by L-arginase. What was selected as a fluorescent dye was 1-aminomethyl-2,3-diazabicyclo[2.2.2]oct-2-ene (101, DBO-amine) and p-sulphonato-calix[4]arene (a cation receptor) was used as the macrocycle, the fluorescence of the DBO-amine 101 being quenched by the calixarene. As a result of the breakdown of the analyte 103, a decrease in fluorescence during the enzymatic reaction was expected and was observed. The calixarene acted as a size-selective receptor which, while maintaining the positive charge, binds the larger guanidium group in the arginine analyte 103 better than the smaller amino group in the ornithine product 101. The schematic representation of the enzyme arginase takes the published crystal structure as a guide (Cama, E et al., Biochemistry 43, 2004, 8987-8999. Protein Data Bank ID: 1T4P) and was produced with the Swiss PdbViewer v3.7 program (http://www.expasy.org/spdbv/).

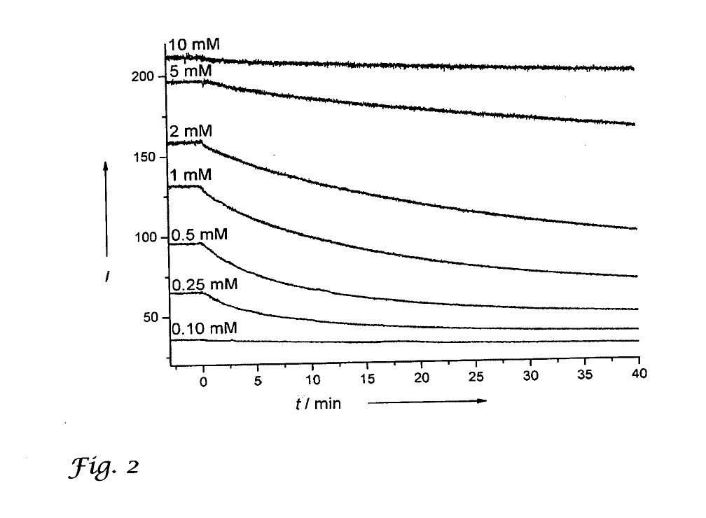

FIG. 2 shows the change in fluorescence following the addition (at time t=0 minutes in each case) of 16 μg/ml of arginase, for different concentrations of arginine. Without any chemical change to the L-lysine, without any radio-active marking, and without any antibodies, the enzymatic reaction could thus be followed easily, directly in solution and without any further incubation stages, with the help of a commercially available fluorescence spectrometer (Varian Cary Eclipse, λ_{exc}=365 nm, λ_{obs}=450 nm in the present case). A clear change in fluorescence, measured as the ratio between the intensity of fluorescence on completion of the reaction and the initial intensity of fluorescence, can already be seen as from an arginine concentration of 0.25 mM. As the amount of arginine increases, so too does the initial intensity, because more of the fluorescent dye is removed from the macrocycle which is quenching the fluorescence. The intensity of the fluorescence on completion of the reaction also increases as the initial concentration of the arginine increases because the product, ornithine, is also able to remove the fluorescent dye (though not as powerfully as the arginine). The reaction was performed in the presence of 100 μM of the fluorescent dye DBO-amine and 200 μM of p-sulphonato-calix[4]arene in water at a pH of 9.5.

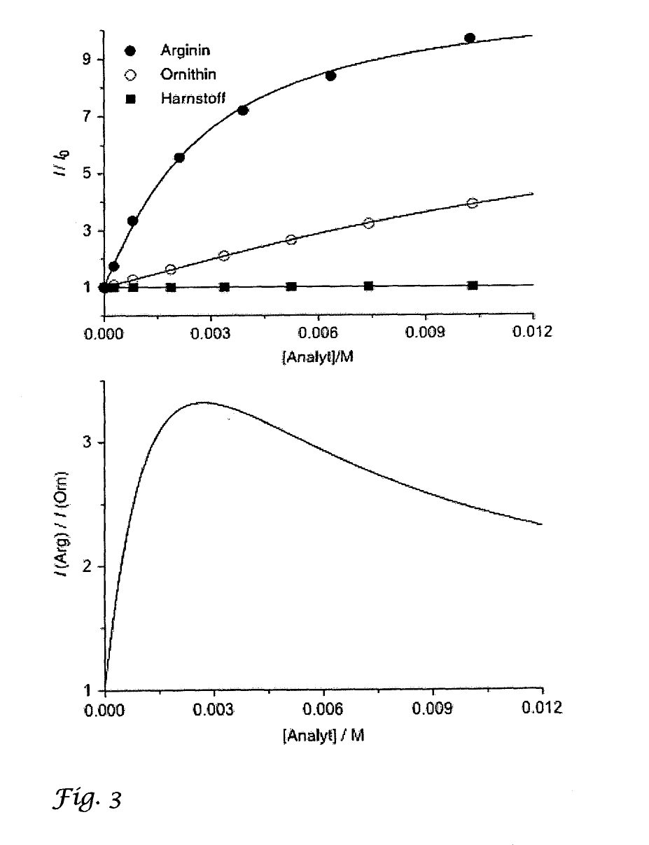

FIG. 3 is a representation of the relationship between the ratios of the initial intensity of fluorescence to the intensity after the complete conversion of L-arginine into L-ornithine in the reaction which is shown in FIGS. 1 and 2. For the same analyte concentration, a removal of the fluorescence dye of a differing extent, and hence an increase in the intensity of fluorescence of a different size, takes place as a function of the binding constants of arginine (K=3170±130 M⁻¹) and ornithine (K=190±70 M⁻¹) to the macrocycle (see the top graph). From the ratio of these intensities of fluorescence (I/Iₒ) can be found the maximum change in the intensity of fluorescence which can be expected during the enzymatic reaction (see the approximated function in the bottom graph). Because of the extremely low binding constant, the binding of the urea (an uncharged molecule having one cation receptor) can be ignored. The binding constants were determined in water at a pH of 7 in the presence of 100 μM of DBO-amine and 200 μM of p-sulphonato-calix[4]arene.

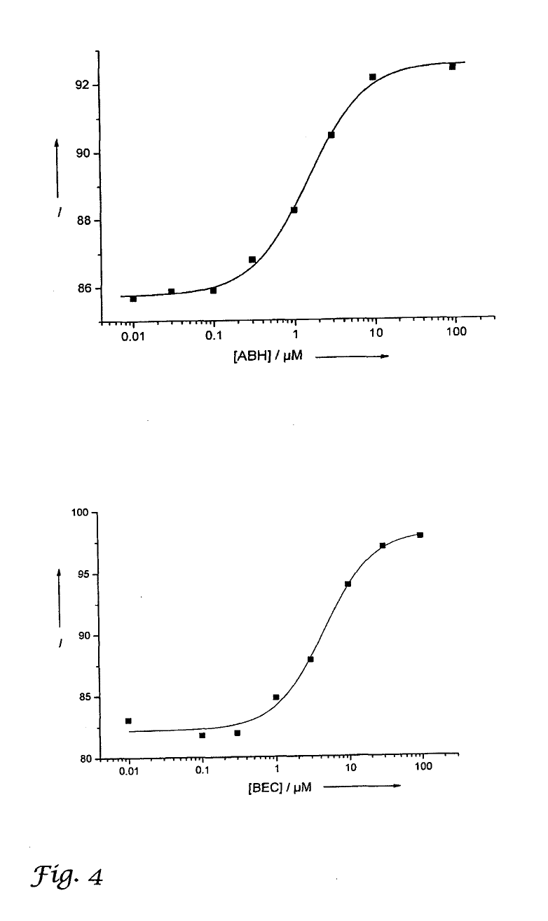

FIG. 4 is a representation of inhibitor screening. A Hill analysis was carried out to determine the effectiveness of the arginase inhibitors a) 2(s)-amino-6-boronohexanoic acid (ABH, Kᵢ=1.0±0.1 μM) and b) S-(2-boronethyl)-L-cysteine (BEC, Kᵢ=4.9±0.5 μM). For this purpose, 0.5 mM of arginine was reacted with 16 μg/ml of arginase in different concentrations of the respective inhibitors. The solution (water, pH 9.5) also contained 100 μM of DBO-amine and 200 μM of p-sulphonato-calix[4]arene. For the Hill analysis, the intensity of fluorescence was plotted against the logarithm of the inhibitor concentration after an incubation time of 6 minutes. The arginase assay is thus suitable for screening a library of inhibitors.

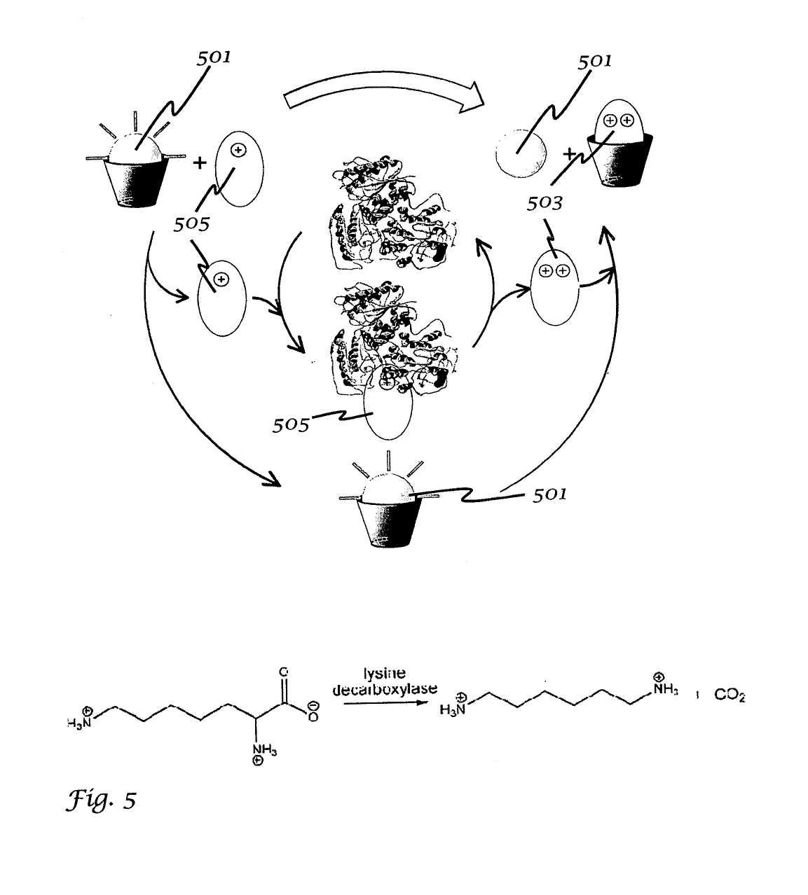

FIG. 5 is a reaction diagram for the conversion of L-lysine (505, analyte precursor) into cadaverine (503, analyte, removes the fluorescent dye) and CO₂ (not shown, most of it escapes from the solution) by L-lysine decarboxylase. Dapoxyl (501) was selected as the fluorescent dye and cucurbit[7]uril (a cation receptor) as the macrocycle, the fluorescence of the dapoxyl (501) being increased by the complexing with cucurbit[7]uril. As a result of conversion of the analyte precursor 505 into the analyte 503, there is a decrease in fluorescence during the enzymatic reaction because more and more dye 501 is removed by the analyte and the increase in the fluorescence of the dapoxyl caused by the macrocycle thus falls off. The cucurbituril acts as a cation receptor which binds the cadaverine analyte 503, which has two positive charges, better than the L-lysine analyte precursor 505, which has a single positive charge (total charge). The schematic representation of the lysine decarboxylase enzyme is again that of the arginase which is used in FIG. 1, because no crystal structure exists for lysine decarboxylase. The representation was produced with the Swiss PdbViewer v3.7 program (http://www.expasy.org/spdbv/).

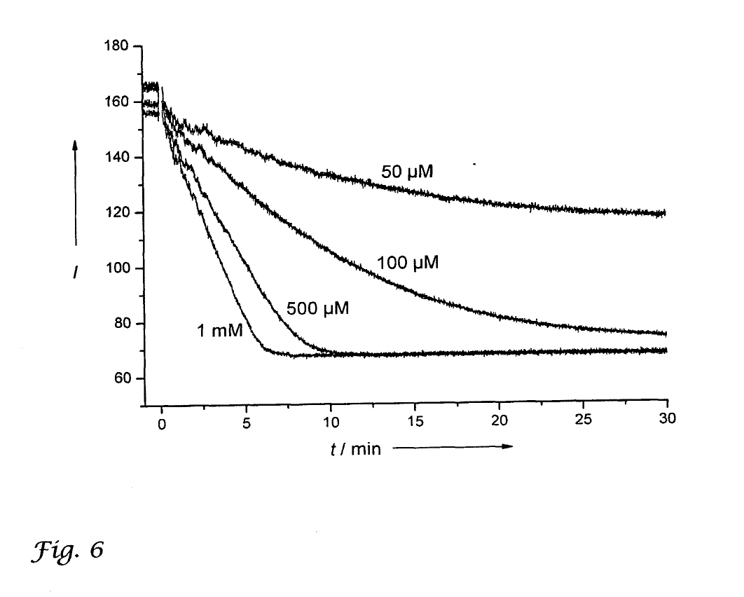

FIG. 6 shows the change in the fluorescence of solutions of different lysine concentrations following the addition (at time t=0 minutes) of 0.4 mg/ml of lysine decarboxylase. The reaction was carried out in the presence of 2.5 μM of dapoxyl and 200 μM of cucurbit[7]uril in a 10 mM NH₄OAc buffer (pH 6.0). Without any chemical change to the L-lysine, without any radio-active marking, and without any antibodies, the enzymatic reaction could thus be followed easily, directly in solution without any further incubation stages, with the help of a commercially available fluorescence spectrometer (Varian Cary Eclipse, λ_{exc}=336 nm, λ_{obs}=390 nm in the present case). It can clearly be seen in this case that the binding constants of lysine (K approx. 2000 M⁻¹) and cadaverine (K>1000000 M⁻¹) are very different. Hence, in the relevant range of concentrations the initial concentration of the lysine has only a very slight effect on the initial intensity of fluorescence. It can also be seen that even 100 μm of cadaverine is enough to remove all the fluorescent dye from the macrocycle, i.e. in the case of 500 μM and 1 mM of lysine only the first 20% and 10% respectively of the reaction could be followed under the given conditions. This is not a problem inasmuch as it is generally only the initial speed as it were of an enzymatic reaction which is determined in order to characterise the reaction. The relatively severe noise during the first 5 minutes after the addition of the enzyme can be attributed to the fact that the lysine decarboxylase used was in the form of an unpurified cell extract and insoluble constituents therefore first has to settle in the buffer.

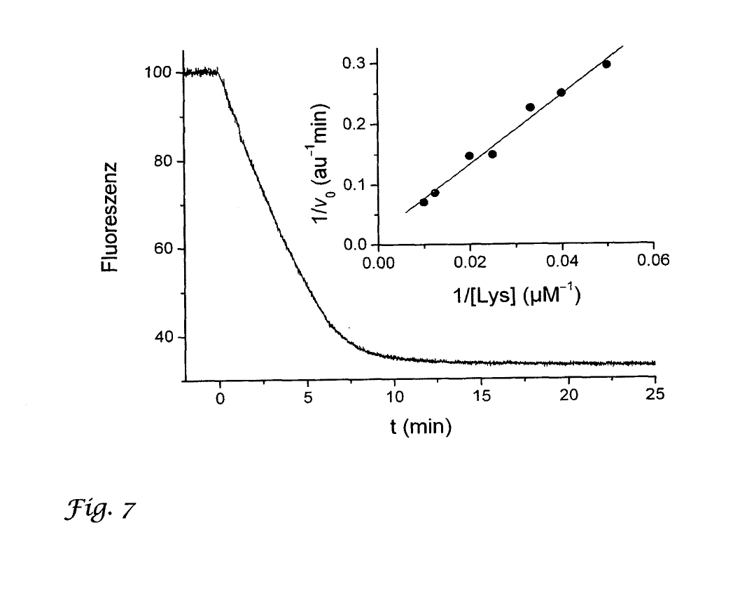

FIG. 7 shows the determination of enzyme kinetics. What is shown by way of example is the change in the intensity of fluorescence of a solution of 100 μM of lysine, 2.5 μM of dapoxyl and 10 μM of cucurbit[7]uril in a 10 mM HN₄OAc buffer (pH 6.0) following the addition (at time t=0 minutes) of 0.04 mg/ml of lysine decarboxylase. By varying the concentration of the substrate (lysine), the kinetic parameters can be determined by the Lineweaver-Burk method. It should be pointed out that the ratio of macrocycle/dye concentrations was changed in comparison with FIG. 6. As a result a smaller amount of enzyme could be used/detected and there was thus a homogeneous solution. What is more, these conditions also allow a 10 times lower concentration of substrate to be used.

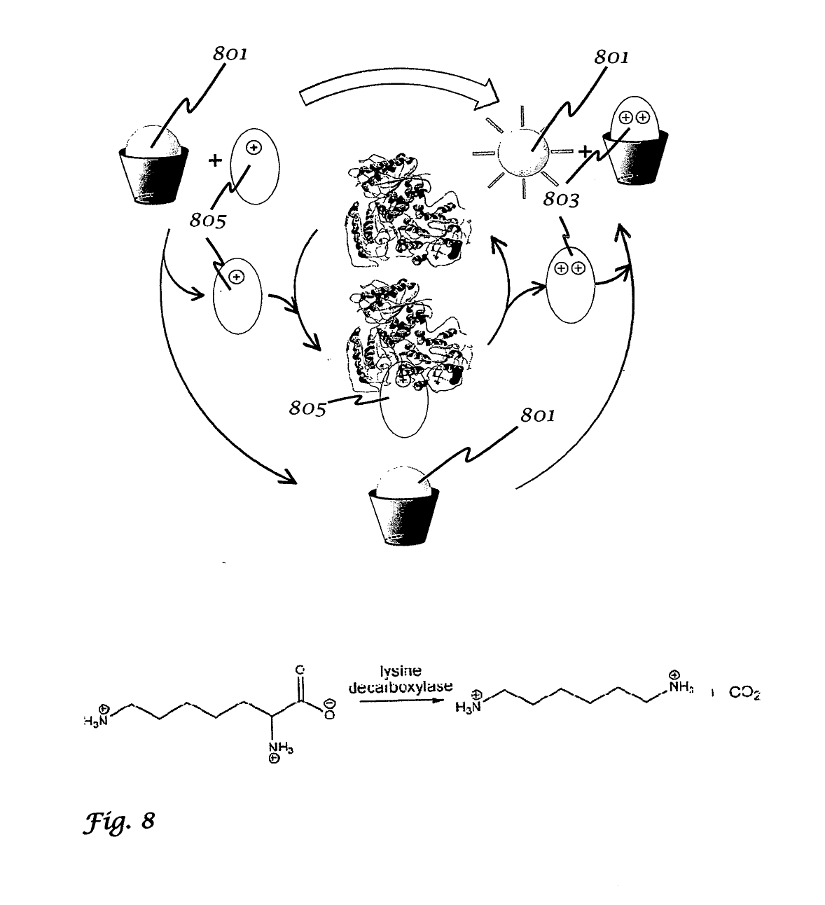

FIG. 8 shows a reaction diagram for the conversion of L-lysine (805, analyte precursor) into cadaverine (803, analyte, removes the fluorescent dye) and CO₂ (not shown, most of it escapes from the solution) by L-lysine decarboxylase. 1-aminomethyl-2,3-diazabicyclo[2.2.2]oct-2-ene (801, DBO-amine) was selected as the fluorescent dye and p-sulphonato-calix[4]arene (a cation receptor) as the macrocycle, the fluorescence of the DBO-amine 801 being quenched by the complexing with the calixarene. As a result of conversion of the analyte precursor 805 into the analyte 803, there is an increase in fluorescence during the enzymatic reaction because more and more dye 801 is removed by the analyte 803 and the quenching of the fluorescence of the DBO-amine caused by the macrocycle falls off. The calixarene acts as a cation receptor which binds the cadaverine analyte 803, which has two positive charges, better than the L-lysine analyte precursor 805, which has a single positive charge (total charge). The schematic representation of the lysine decarboxylase enzyme is again that of the arginase which is used in FIG. 1, because no crystal structure exists for lysine decarboxylase. The representation was produced with the Swiss PdbViewer v3.7 program (http://www.expasy.org/spdbv/).

This example illustrates the wide applicability, because in what follows evidence will be shown of the same reaction as in FIGS. 5 to 7 but with a different system of macrocycle and fluorescent dye.

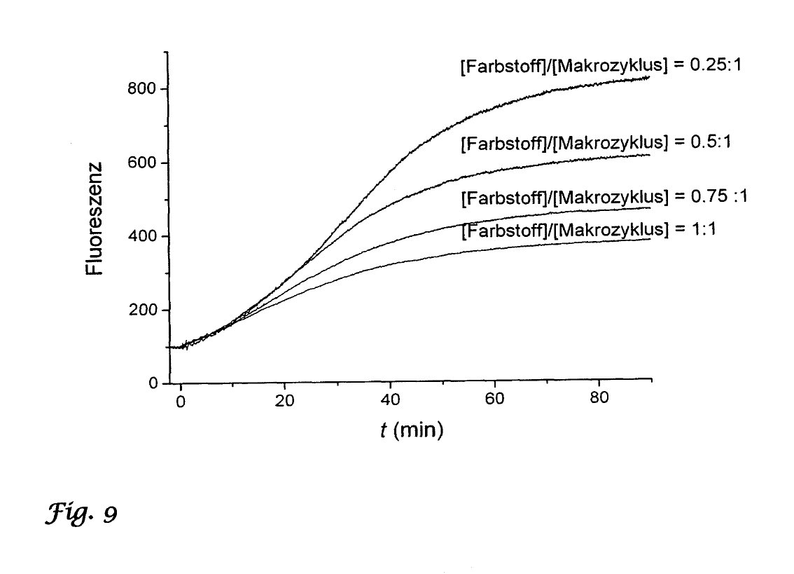

FIG. 9 shows the change in the fluorescence of solutions having different concentration ratios of dye/macrocycle, following the addition (at time t=0 minutes) of 0.4 mg/ml of lysine decarboxylase. The reaction was carried out in the presence of 100-400 μM of DBO-amine and 400 μM of calixarene in a 10 mM NH₄OAc buffer (pH 6.0). The concentration of the substrate was 500 μM. It can clearly be seen in this case that different experimental conditions may result in unusual curves. In this way, the intensity of fluorescence is not a direct reflection of (is not proportional to) the enzymatic conversion but is only a qualitative indication of the activity of the enzyme. On the other hand, it does demonstrate that the increase in the intensity of fluorescence for example can be increased to a greater degree by varying the defining conditions.

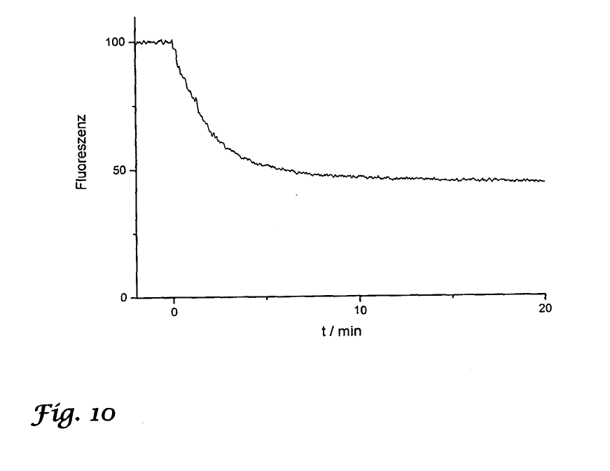

FIG. 10 shows the possibility of examining unpurified cells rather than a purified enzyme. What is shown by way of example is the change in the intensity of fluorescence of a solution of 150 μM or tyrosine, 2.5 μM of dapoxyl and 10 μM of cucurbit[7]uril in a 10 mM NH₄OAc buffer (pH 6.0) following the addition (at time t=0 minutes) of 0.04 mg/ml of dried Streptococcus faecalis cells. The cells contained the enzyme tyrosine decarboxylase, which catalyses the conversion of tyrosine into tyramine.

1. Method of determining, in a specimen, a change in the concentration of an analyte, and in particular of determining changes in concentration in an enzymatic reaction, comprising the steps of:
a) providing a fluorescent dye and a macrocycle for the specimen to be examined, the analyte, in the range of concentrations of the analyte which are to be examined, removing the fluorescent dye from the macrocycle, and
b) measuring a fluorescent property of the fluorescent dye at least two points in time.

2. Method according to claim 1, also comprising
a co-factor for the conversion of the analyte, so that, in the range of concentrations of the analyte which are to be examined, the converted analyte does not remove the fluorescent dye from the macrocycle or does not do this as powerfully as the analyte, and/or
a precursor (educt) of the analyte, which is converted into the analyte and which, in the range of concentrations of the analyte which are to be examined, does not remove the fluorescent dye from the macrocycle or does not do this as powerfully as the analyte.

3. Method according to claim 2, characterised in that the co-factor is a catalyst.

4. Method of screening for finding a co-factor for an analyte, comprising the steps of:
a) providing a plurality of mixtures containing the preselected analyte, a co-factor to be tested for the analyte, a fluorescent dye, and a macrocycle, the analyte, in the range of concentrations of the analyte which are to be examined, removing the fluorescent dye from the macrocycle, and,
b) performing a method of determination according to one of the foregoing claims with each of the mixtures provided in a).

5. Method of screening for finding an inhibitor of a preselected reaction of an analyte, comprising the steps of:
a) providing a plurality of mixtures containing the preselected analyte and the co-factor which is required for the carrying out of the preselected reaction, an inhibitor, which is to be tested, of the reaction, a fluorescent dye, and a macrocycle, the analyte, in the range of concentrations of the analyte which are to be examined, removing the fluorescent dye from the macrocycle, and,
b) performing a method of determination according to one of the foregoing claims with each of the mixtures provided in a).

6. High-throughput method of screening according to either of claims 4 and 5, characterised in that at least 48 mixtures are provided in step a).

7. Apparatus for determining a change in the concentration of an analyte, comprising
a specimen containing a fluorescent dye and a macrocycle, the analyte, in the range of concentrations of the analyte which are to be examined, removing the fluorescent dye from the macrocycle, and
a co-factor for converting the analyte, so that, in the range of concentrations of the analyte which are to be examined, the converted analyte does not remove the fluorescent dye from the macrocycle or does not do this as powerfully as the analyte, and/or
a precursor (educt) of the analyte, which is converted into the analyte and which, in the range of concentrations of the analyte which are to be examined, does not remove the fluorescent dye from the macrocycle or does not do this as powerfully as the analyte,

8. Apparatus according to claim 7, also comprising a light source for exciting the fluorescent dye and a detector for measuring the fluorescence emanating from the fluorescent dye and/or the light absorption of the fluorescent dye.

9. Apparatus according to either of claims 7 and 8, also comprising a carrier having receptacles for at least 48 specimens to be examined.

10. Use of a fluorescent dye and a macrocycle to determine a change in the concentration of an analyte.

11. Use according to claim 10 of an anion-binding macrocycle, and preferably a calixarene having cationic groups, together with a fluorescent dye, for detecting and/or determining a change in the concentration of an anion.

12. Use according to claim 10 of a cation-binding macrocycle, and preferably a calixarene having anionic groups, and/or of a cucurbituril, together with a fluorescent dye, for detecting and/or determining a change in the concentration of a cation.

13. Use according to claim 10 of a hydrogen-bridge forming macrocycle, and preferably a cyclodextrin, together with a fluorescent dye, for detecting and/or determining a change in the concentration of a hydrogen-bridge forming analyte.

14. Use according to claim 10 of a stereo-selective macrocycle, and preferably a cyclodextrin, together with a fluorescent dye, for detecting and/or determining a change in the concentration of a stereo-isomer of an analyte.
